(19) United States
(12) Patent Application Publication
(10) Pub. No.: US 2009/0036402 A1
(43) Pub. Date: Feb. 5, 2009

(54) Use of compounds from centella asiatica

(76) Inventors: Gerard Sene, Paris (FR); Alain Loiseau, Arzacq (FR); Jean-Christophe Lepetit, Colombes (FR)

Correspondence Address: BAYER HEALTHCARE LLC;CONSUMER CARE DIVISION, 36 COLUMBIA ROAD, MORRISTOWN, NJ, 07962, US

(21) Appl. No.: 12/151,697

(22) Filed: May 8, 2008

(30) Foreign Application Priority Data
Nov. 9, 2005 (EP) 05292384.4
Oct. 27, 2006 (EP) PCT/EP2006/010355

(51) Int. Cl. A61K 31/715; A61K 31/19; A61P 17/00

(52) U.S. Cl. 514/54; 514/557

(57) ABSTRACT

Use of compounds extracted from Centella Asiatica in topical and other formulations for skin care.

### 1. FIELD OF THE INVENTION

This invention relates to the use of compounds from Centella Asiatica in cosmetics, pharmaceuticals and food supplements for improving keratinocytes differentiation and for enhancing the epidermal functionality.

### 2. BACKGROUND OF THE INVENTION

Centella Asiatica, known as Violette marronne on Reunion Island, Gotu Kola or Indian pennywort in India, Centella repanda in North America, and Talapetraka in Madagascar, is a polymorphous herb and belongs to the family of Umbelliferae (Apiaceae), particularly to the Hydrocotyle subfamily. It grows wild throughout the tropics and prefers moist and shady regions at an altitude of about 600 to 1200 meters above sea level. Centella Asiatica has three varieties: Typica, Abyssinica, and Floridana. The herb is known and used for its healing, sedative, analgesic, antidepressant, antiviral and antimicrobial properties. The biological activity of the herb appears to be due to the presence of triterpene molecules (shown below) such as Asiaticoside (I), Madecassoside (under its 2 isomeric forms: madecassoside itself and terminoloside) (II), Asiatic acid (III) and Madecassic Acid (IV). These triterpenes contribute to the natural defense of the herb against bacterial attack due to the anti-bacterial properties of the genins (III, IV), which may be obtained by hydrolysis from the heterosidic reserve forms (I, II).

Extracts containing these triterpene compounds have been used in the pharmaceutical and cosmetic industry for skin care, for treating skin diseases, for treating wounds, for healing scars, for venous insufficiency, for dermis restoration, and for anti-inflammatory purposes as disclosed in WO2004/062678.

Various forms of the triterpene series are as shown below

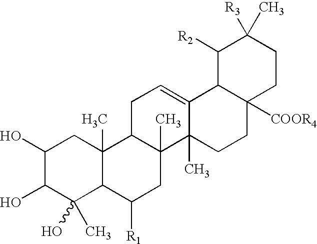

| | | Ligand | | | |
|---|---|---|---|---|---|
| Form | Name | R₁ | R₂ | R₃ | R₄ |
| I | Asiaticoside | —H | —CH₃ | —H | -Glu-Glu-Rha |
| II | Madecassoside | —OH | —CH₃ | —H | -Glu-Glu-Rha |
| | Terminoloside | —OH | —H | —CH₃ | -Glu-Glu-Rha |
| III | Asiatic Acid | —H | —CH₃ | —H | —H |
| IV | Madecassic Acid | —OH | —CH₃ | —H | —H |

In the table above, C, H, and O stand for carbon, hydrogen and oxygen, respectively. Glu means glucose, and Rha means rhamnose.

Aquaporins were discovered in 1988. Aquaporins serve as water channels and increase the permeability of membranes to water for as much as ten-fold. They are located in basal layers of epitheliums of numerous organs like kidney, lung or intestines. Takata K, et al., Prog Histem Cytochem, vol. 39 p 1-835 (2004). These transmembrane proteins serve to facilitate water transport through the skin down osmotic gradients that have low activation energy. Eleven aquaporins have been identified in mammals to date.

Aquaporin-3 (AQP-3) is unique in its structure, having the lowest homology with other aquaporins. Ishibashi, K; et al., American Journal Of Physiology, 272 (2) 2, p. F235-F241 (1.997). AQP-3 transports glycerol and water (aquaglyceroporin) and is regulated by p 73. Zheng, X.; et al., FEBS Letters, 489 (1) p. 4-7 (Jan. 26, 2001). AQP-3 expression begins relatively late in fetal life. Matsuzaki, T.; et al., J. Histochemistry and Cytochemistry: Official Journal of the Histochemistry Society, 47 (10), p. 1275-1286 (1999). AQP-3 is generally found in keratinocytes, especially so in skin. Sougrat R., et al. J. Invest. Dermatol., vol. 118, p. 678-85 (2002). The abundance of basolateral AQP-3 in epithelial tissue and its expression in many non-epithelial cells suggest that AQP-3 is a major participant in osmolyte homeostasis in the human body and may play an important role in the hydration of the skin. Mobasheri, A; et al., J. Mol. Histol., vol. 36, 1-2, p. 1-14 (2005).

Several studies on mice deficient in AQP-3 illustrate the influence of AQP-3 on skin. SHK1 mice have been shown to have dry skin showing reduced stratum corneum hydration, decreased elasticity, and impaired functionality. Hara, M., et al., J. Biol. Chem., 277, (48), p. 46616-46621 (2002). AQP-3 has been theorized to by a “water-clamp” that improves the hydration of the epidermis below the stratum corneum. Sougrat R., et al., J. Investigative Dermatology, 118 (4), p. 678-685 (2002). A more recent study suggests that glycerol transport, rather than water transport, may be a more important function of AQP3 in skin physiology because the glycerol content of AQP-3 deficient mice is only reduced in the stratum corneum and in the epidermis. Hara et al., Biology Of The Cell/Under The Auspices Of The European Cell Biology Organization, 7 (97), p. 479-486 (2005). Accordingly, the dry and relatively inelastic skin in AQP-3 null mice may be related to the humectant properties of glycerol, and the impaired stratum corneum repair observed in null mice may be due to impaired epidermal biosynthetic function.

Moreover, AQP-3 has been implicated in the triglyceride content of the epidermis and contributes partially to the lipid composition of the stratum corneum and ceramides. The water and associated transfer of electrolytes is beneficial for cell communication, reparation and drainage. Therefore AQP-3 regulating factors may also be helpful for the treatment of skin diseases associated with altered skin, water content, lipidic disorders and cells communication dysfunctions as discussed in WO 2001/37799.

Functionality of the epidermis can also be altered by an impairment of the stratum corneum organization and more generally by a poor differentiation of keratinocytes leading to a decrease of the skin barrier function. Genetic or chronic deficiencies on materials such as ceramides, filaggrin, pro-filaggrin, natural moisturizing factor, transglutaminase and other materials can leas to less than optimal differentiation and a deficient maturation of keratinocytes.

Filaggrin, in particular, is an important ingredient in the organization of lamellar corneocytes and, consequently, in the maintenance of skin water content, and the biological and physical integrity of the skin. Filaggrin is a basic, histidine-rich protein synthesized by cells of keratinizing epithelia that support major physiological functions in epidermis. The protein precursor of filaggrin, profilaggrin, constitutes a major component of keratohyalin granules in the epidermal granular layer. Kanitakis J., et al., Pathol Anat Histopathol., 412 (4), p. 375-82 (1988). Obtained by dephosphorylation of profilaggrin, the filaggrin filament-associated protein aggregates keratin in the stratum corneum of mammalian epidermis during the terminal differentiation of keratinocytes into comeocytes. It therefore plays a role in the organization of lamellar corneocytes, in cohesion of cells, in stratum corneum resistance, in flexibility, and in epidermal barrier function.

Transglutaminases are another class of compounds having epidermal functionality. These compounds play in important role in mature keratinocytes. Transglutaminases (TGMs) are a widely distributed group of calcium-dependent enzymes that catalyze formation of covalent isopeptide bonds. The cross-linked products of the operation of these enzymes are highly resistant to mechanical challenge and proteolytic degradation. Griffin, M., et al., Biochem J, 368, p. 377-96 (2002). Four out of the nine TGMs discovered to date are expressed in the epidermis. These enzymes have been shown to be expressed maximally in the upper spinous and granular cells of the epidermis. Thacher, S. M. et al., Cell 40, p. 685-695 (1985), Parenteau N., et al, Differentiation, 33, p. 130-141 (1986).

TGMs are particularly useful in the terminal differentiation of the epidermis, where they heavily cross-link keratins and a range of differentiation-specific structural proteins (such as involucrin, loricrin, and others). TGMs are also useful in the formation of the cornified cell envelope in the biogenesis of the stratum corneum (Kalinin, A. et al., Bioessays, 24, p. 789-800 (2002), Ta B., et al., J Cell Sci 95 p. 631-638 (1990)). Mutations of TGMs and their substrates can cause severe skin diseases. For example, dysfunction of TGM-1 has been shown to cause lamellar ichthyosis, a disease characterized by excessive scaling and shedding of the outer epidermis (Cassidy, A. et al., Am. J. Hum. Genet., 77, p. 909-917 (2005), Candi, E, et al., Nat Rev Mol Cell Biol, 6, p. 328-340 (2005)).

Despite the recent advances in knowledge in the understanding of skin biology, a continuing need remains for compositions and formulation that can provide ongoing benefit to the skin.

### SUMMARY OF THE INVENTION

The principal object of the invention therefore is to provide a composition for treating skin for various conditions and diseases.

Additional objects and advantages of the invention will be set forth in part in the description that follows, and in part will be obvious from this description, or may be learned by practice of the invention. The objects and advantages of the invention may be realized and attained by means of the instrumentalities and combinations particularly pointed out in the appended claims.

To achieve the foregoing objects and in accordance with the purpose of the invention, as embodied and broadly described herein, the invention provides a composition that comprises at least one compound from Centella Asiatica. This compound may be selected from among madecassoside, terminoloside, asiaticoside, madecassic acid, asiatic acid and mixtures thereof.

An advantage of the composition is that the compound found in the composition may serve to activate at least one of AQP-3, filaggrin or transglutaminase in the skin of an animal, such as a human being. The activation of these materials can improve the health of the skin.

### DETAILED DESCRIPTION OF THE PREFERRED EMBODIMENTS OF THE INVENTION

Reference will now be made in detail to the presently preferred embodiments of the invention.

The invention contemplates the use of at least one compound selected from the group consisting of madecassoside, terminoloside, asiaticoside, madecassic acid and asiatic acid. The compound is believed to improve the differentiation of keratinocytes and to enhance epidermal functionality. Without being limited by theory, it is believed that the compounds work to improve or activate the functioning of AQP-3, filaggrin, transglutaminase or some combination of these materials.

The active compounds according to the invention can be found in various plants, most preferably in the plant Centella Asiatica. Extracts from plants that contain the active compounds of the invention can be obtained, for example, by extraction as described in PCT Application Publication Number WO2004/062678 to Loiseau Alain et al., published on Jul. 29, 2004, for Centella Asiatica. The extracts of Centella Asiatica may comprise different pentacyclic triterpenes such as asiatic acid, asiaticoside, madecassic acid, madecassoside and/or terminoloside. These compounds can be isolated from the extracts using known techniques, such as those used to isolate the compounds from extracts of Centella Asiatica as described in WO2004/062678. The compounds of the invention can be used as a single isolated substance, in combination with one or more other compounds of the invention, or as the plant extract, so long as the extract comprises one or more of the compounds of the invention.

Preferred compounds include madecassoside, terminoloside, and asiaticoside. More preferred compounds are madecassoside and terminoloside. Plant extracts containing the preferred and more preferred compounds are also preferred. Such plant extracts may contain any amount of the compounds, but preferably the compounds comprise at least about 75% by weight, and more preferably at least about 85% by weight of the total plant extract. Other ingredients in the plant extracts may include fatty acids. When the plant extract comprises more than one of the compounds, or when the invention is formulated with a blend of previously isolated compounds, the weight ratio of asiaticoside, if any, to the combined weight of madacassoside and terminoloside, if any, may be any amount. Preferred weight ratios, however, may be from about 5:95 to about 25:75. The weight ratio of madecassoside and terminoloside may also be any amount, but preferably may be from about 30:70 to about 70:30, and more preferably from about 40:60 to about 60:40 by weight.

The purity of the mixture is preferably greater than 95% relative to the total weight of the mixture. More preferably the mixture contains no or only in trace amounts of asiaticoside.

If the materials are to be employed in liquid form, a preferred solvent comprises a mixture of water and an alcohol, preferably ethanol. The ratio of the volume of water to the volume of alcohol is preferably from about 50:50 up to about 90:10, and more preferably about 75:25.

Compounds, mixtures and extracts of the present invention can be administered in any form by any effective route, including, without limitation, oral, parenteral, enteral, intravenous, intraperitoneal, topical, transdermal (for example using conventional transdermal patch technology), ophthalmic, nasal, local, non-oral (for example aerosol, inhalation, subcutaneous, intramuscular, buccal, sublingual, rectal, vaginal, intra-arterial, and intrathecal). The compounds can be administered alone or in combination with other, active or inactive ingredient(s). Topical administration is most preferred.

Compounds, mixtures and extracts of the present invention may be placed into conventional cosmetic or pharmaceutical preparations and may also be uses as a food supplement or additive. These preparations include liquid or solid formulations such as coated or uncoated tablets, capsules, pills, powders, granules, elixirs, tinctures, solutions, suspensions, suppositories, syrups, solid and liquid aerosols, emulsions, pastes, creams, ointments, milks, gels, salves, serums, foams, shampoos, sticks or lotions.

An aqueous solution is the preferred preparation, for example as a cosmetic white or colored cream, ointment, milk, gel, salve, serum, foam, shampoo, stick, cream, paste, or lotion.

Compounds, mixtures and extracts of the present invention may be combined with any other suitable additive or pharmaceutically acceptable carrier, including those described in Remington: The Science and Practice of Pharmacy (Gennaro and Gennaro, eds, 20th edition, Lippincott Williams & Wilkins, 2000); Theory and Practice of Industrial Pharmacy (Lachman et al, eds., 3rd edition, Lippincott Williams & Wilkins, 1986); and Encyclopedia of Pharmaceutical Technology (Swarbrick and Boylan, eds., 2nd edition, Marcel Dekker, 2002). These additives are referred to herein as “pharmaceutically or cosmetically acceptable carriers” to indicate they may be combined with the active drug and may be administered safely to a subject for therapeutic purposes.

The dosage of the compounds, mixtures and extracts of the present invention depends on the type and nature of problem to be treated or prevented by administration of the compounds and the vehicle for delivering the compounds. The amount of compound administered can vary widely depending on considerations such as the particular compound and dosage unit employed, the mode and time of administration, the period of treatment, the age, sex, and general condition of the patient treated, the nature and extent of the condition treated, the rate of drug metabolism and excretion, the potential drug combinations, drug-drug interactions, and other considerations that will be well understood by those skilled in the art.

Compositions comprising the active compounds, mixtures and extracts of the present invention in an amount of from about 0.005% to about 10% by weight of the total composition are preferred, more preferably from about 0.01% to about 5% by weight, and most preferably from about 0.1% to about 3% by weight.

The pharmaceutical or cosmetic composition of the invention may administered as needed, but preferably one or more, more preferably up to three, most preferably up to two times per day.

In some cases be advantageous to deviate from the amounts set froth herein, depending on body weight, individual reaction to the active ingredient, type of preparation and time or interval over which the administration is effected. For instance, less than the minimum amounts set forth above may be sufficient in some cases, while the upper limit specified may be exceeded in other cases. For administration of relatively large amounts, it is preferred that these doses be divided into several smaller individual doses administered over the course of a day.

Administration of the compounds, mixtures or extracts of the present invention can maintain good skin health or prevent or treat skin diseases or conditions. For example, administration of the compounds, mixtures or extracts of the invention in any appropriate format can increase the expression of AQP-3 in the epidermis, including the living layers (basal keratinocytes, stratum granulosum and spinosum), the expression of filaggrin, and the expression of transglutaminase. Compounds, mixtures or extracts of made in accordance with the invention can therefore be used for any disease or disorder associated with a deficiency of AQP-3, filaggrin or transglutaminase expression. The compounds, mixtures or extracts of the invention may maintain or improve the hydration of the skin, preferably hydration of the epidermis below the stratum corneum. The elasticity and/or epidermal resistance of the skin can also be maintained or restored. The lamellar organization of corneocytes can be improved and the cohesion of stratum corneum, epidermal barrier function, and skin impermeability can be increased, either alone or in combination. Epidermal dehydration can be prevented and the regeneration of the protective lipid film of the skin can be supported. The electrolyte and water transfer of the skin can be improved. Osmolytic homeostatis can be activated and/or regenerated. The epidermal detoxification, the communication of keratinocytes and the reparation of the membrane of keratinocytes can be improved. The drainage of keratinocytes can be enhanced. Compounds, mixtures and extracts of the present invention can be used for improving the differentiation of keratinocytes, for enhancing the epidermal functionality and/or for treating or preventing of skin diseases or skin disorders such as epithelial lesions, atrophy, ulceration, hyperplasia, dysplasia, cutaneous squamous lesions, dry skin, dry atopic dermatitis, ichtyosic skins or atopic pytiriasis. Compounds, mixtures and extracts of the present invention can also be used for the treatment and/or prevention of abnormalities of epidermal maturation and/or keratinization.

Compounds, mixtures and extracts of the present invention are preferably used for activating AQP-3 e.g. in different epidermal layers. The transfer of water, water-soluble compounds such as electrolytes or neutral solutes, and the transfer of glycerol can be increased by such activation, which results in activation or regeneration of osmolyte homeostasis and enhancement of the keratinocytes drainage, which improves cell detoxification, assists in keratinocyte membrane repair, helps with treatment of lipidic disorders, and improves keratinocyte communication (between cells and between epidermis on the one hand and the Epidermal Dermal Junction or the dermis on the other hand).

Compounds, mixtures and extracts of the invention are also preferably used for activating filaggrin. This activation affects the differentiation of keratinocytes, so abnormalities of epidermal maturation and/or keratinization can be treated, cohesion of stratum corneum (corneocytes lamellar organization), the epidermal barrier function and skin impermeability can be increased, epidermal dehydration can be prevented, and the epidermal elasticity/flexibility and resistance may be improved.

Compounds, mixtures and extracts of the present invention may also preferably be used for activating transglutaminase. This activation affects the maturation of keratinocytes, which can help treat abnormalities of epidermal maturation and/or keratinization, help maintain the cohesion of the stratum corneum (corneocytes lamellar organization), increase the epidermal barrier function, and increase skin impermeability to improve epidermal resistance.

The invention will be further appreciated by consideration of the following examples:

### Example 1

### Evaluation of Keratinocytes AOP-3 Expression

Biopsies from abdominal plastic surgery from a 41-year-old woman were used in this ex vivo experiment. The biopsies were cultured in survival explants medium BEM (BIO-EC's Explants Medium).

Four milligrams of a formulation containing 3% of a mixture of madecassoside and terminoloside was applied to a cultured biopsy sample. A control biopsy sample, treated with a solution containing only the excipient, without the compounds of the invention, was also prepared. The administration was carried out once per day at days D0, D1, D2, D3, and D4.

Histological studies were performed at 0 hours, 3 hours, 24 hours and 5 days. The samples were prepared by dehydrating explants and impregnating the explants with paraffin. The explants were then fixed with Bouin's solution. They were then cut and stained by Masson's trichome stain. Flurorescent immunomarking was then carried out. Aquaporin-3 (AQP-3) was marked on frozen cryostat cut tissues with anti-AQP3 from Chemicon (polyclonal ref AB3276) with a biotin/streptavidin system and revealed by FITC. Observations were done by electronic microscope and quantification by image analysis. The results are shown in Table 1.

TABLE 1
Observations of the expression of AQP 3 in the epidermic layers

Time

| | | | |
|---|---|---|---|
| 0 hours | 3 hours | 24 hours | 5 days |

Stratum corneum basis

| | | | | |
|---|---|---|---|---|
| Control | 0 | X | X | X |
| Excipient | X | 0 | 0 | 3 |
| Excipient plus | X | 0 | 1 | 4 |
| 3% mixture | | | | |

Basal poles of basal keratinocytes

| | | | | |
|---|---|---|---|---|
| Control | 0 | X | X | X |
| Excipient | X | 0 | 0 | 3 |
| Excipient plus | X | 0 | 4 | 4 |
| 3% mixture | | | | |

Key: X means not measured, 0 means absent, 1 means weak, 2 means very moderate, 3 means moderate, 4 means clear, 5 means very clear, and 6 means high.

At 0 hours, the AQP-3 expression is clearly visible in membranes (regular, pericellular presence). AQP-3 expression is absent at the base of the stratum corneum, clear-cut in the epidermal upper layers, moderate in the basal layer and absent in the basal pole of basal keratinocytes (usually an area lacking AQP-3). A time-dependent phenomenon was observed for the increase of AQP-3 expression in epidermis by administration of the mixture of the invention: AQP-3 was more clearly visualized after 3 hours, and even more so after 24 hours. After 5 days an important development of the protein channels is observed at the basal pole of the basal keratinocytes. This particular location of AQP-3 is likely a favorable factor for the dermoepidermal communication, especially for the heme originally from vascularization, which is a known differentiation inductor.

The quantification of the expression of AQP 3 in the epidermic layers is shown in Table 2 as a percentage of the surface occupied by the aquaporin 3 measured at day 5

TABLE 2
Percentage of Surface Occupied by AQP-3 at 5 Days

| | | | In the basal pole of the | |
|---|---|---|---|---|
| | In the living epidermis | | basal keratinocytes | |
| | Average | Deviation | Average | Deviation |
| Excipient | 42.9 | 1.9 | 31.1 | 1.8 |
| Excipient plus 3% | 56.9 | 3.1 | 44.9 | 3.7 |
| mixture | | | | |

On Day 5, the formulation containing 3% of a mixture of Madecassoside—Terminoloside significantly increased the AQP-3 content by 33% in the living epidermis and by 44% in the basal pole of the basal keratinocytes layer when compared to a formulation containing only the excipient.

### Example 2

### Evaluation of Keratinocytes Filaggrin Synthesis

Biopsies from abdominal plastic surgery from a 41-year-old woman were used in this ex vivo experiment. The biopsies were cultured in survival explants medium BEM (BIO-EC's Explants Medium).

Four milligrams of a formulation containing 3% of a mixture of madecassoside and terminoloside was applied to a cultured biopsy. A control formulation containing only excipients without the active compounds was applied to another cultured biopsy. Administration was carried out once per day at days D0, D1, D2, D3, and D4.

Histological studies were performed after 0 hours, 3 hours and 24 hours. Explants were dehydrated and impregnated with paraffin and then were fixed with Bouin's solution. The explants were then cut and stained by Masson's trichome stain. Fluorescent immunomarking was carried out by marking Filaggrin on frozen cryostat cut tissues with anti-filaggrin from BTI Cliniscience (clone OKTB1 ref BT 576) with a biotin/streptavidin system and revealed by FITC. Observations were done by electronic microscope and quantification by image analysis.

The expression of AQP 3 in the epidermic layers was observed. At 0 hours, filaggrin was normally expressed at the stratum corneum basis. After 3 hours an increased expression was observed. After 24 hours, an increased fluorescence was observed in a greater number of cell layers. Marking was present in 9 to 10 layers from the stratum corneum basis.

The cohesion of the stratum corneum stratification was improved due to filaggrin. Its increased concentration in the upper granular layer leads to an enhancement of the organization and physical quality in the stratum corneum (apoptotic regulation) and a strengthening of the hydric affinity following its hydrolysis. Anti-dryness activity can thereby be improved.

The percentage of the surface occupied by filaggrin in the epidermis at after 24 hours is shown in Table 3.

TABLE 3
Percentage of Surface Occupied by Filaggrin

| | 0 hours | | 24 hours | |
|---|---|---|---|---|
| | Average | Deviation | Average | Deviation |
| Blank | 9.8 | 1.4 | | |
| Excipient | | | 5.9 | 1.8 |
| Excipient + 3% mixture | | | 9.4 | 1.7 |

On day 1 the formulation containing 3% of a mixture Madecassoside—Terminoloside significantly increased the filaggrin content of epidermis by 59% when compared to the excipient.

### Example 3

### Evaluation of Keratinocytes Trans Glutaminase Synthesis

Biopsies from abdominal plastic surgery from a 41-year-old woman were used in this ex vivo experiment. The biopsies were cultured in survival explants medium BEM (BIO-EC's Explants Medium).

Four milligrams of a formulation containing 3% of a mixture of madecassoside and terminoloside was applied to a cultured biopsy. A control formulation containing only excipients without the active compounds was applied to another cultured biopsy. Administration was carried out once per day at days D0, D1, D2, D3, and D4.

Histological studies were performed after 0 hours, 3 hours and 24 hours. Explants were dehydrated and impregnated with paraffin and fixed with Bouin's solution. The explants were then cut and stained by Masson's trichome stain. Fluorescent immunomarking was carried out by marking transglutaminase on frozen cryostat cut tissues with anti-membrane transglutaminase from Harbor Bio-products (MAB Clone B.C1) with a biotin/streptavidin system and revealed by FITC. Observations were done by electronic microscope and quantification by image analysis.

On day 0, the transglutaminase expression is light and irregular. After 24 hours, the immunostaining was clear on explants receiving 3% of the Madecassoside—Terminoloside blend. The immunostaining was clearly lighter on those explants receiving the excipient. The percentage of the surface occupied by transglutaminase in the epidermis at 0 hours and after 24 hours is shown in Table 4.

TABLE 4
Percentage of Surface Occupied by Transglutaminase

| | 0 hours | | 24 hours | |
|---|---|---|---|---|
| | Average | Deviation | Average | Deviation |
| Blank | 0.73 | 0.51 | | |
| Excipient | | | 0.02 | 0.02 |
| Excipient + 3% mixture | | | 0.20 | 0.14 |

On day 1, the formulation containing 3% of a mixture Madecassoside—Terminoloside moderately, but significantly, increased the transglutaminase compared to the formulation containing only the excipient.

### Example 4

### Typical Composition of a Cream

Creams may be prepared having the formulations set forth in Table 5.

TABLE 5
Ingredients of Creams (INCI, w/w %)

| Ingredient | Cream 1 | Cream 2 |
|---|---|---|
| Mixture of madecassoside and terminoloside or | 1.0% | 1.0% |
| extract of Centella Asiatica | | |
| Aluminium starch octenyl succinate | | 2.0% |
| Beheneth-10 | 1.5% | |
| Beheneth-25 | 1.5% | |
| Behenyl alcohol | 2.0% | |
| Butylene glycol | 2.0% | |
| Caprylic/capric triglyceride | | 5.0% |
| Carbomer | 0.2% | 0.3% |
| Cetearyl glucoside and cetearyl alcohol | | 5.0% |
| Cetearyl isononaoate | 5.0% | 3.0% |
| Dimethicone | 1.0% | |
| Dimethicone crosspolymer | | 2.0% |
| Dycaprylyl carbonate | 5.0% | 5.0% |
| Glycerol | 3.0% | 2.0% |
| Hexyl laurate | 5.0% | |
| Hydrogenated vegetable glycerides | 2.0% | |
| Isohexadecane | 5.0% | |
| Parabens | | 0.1% |
| PEG 32 | | 2.0% |
| Phenoxyethanol and parabens | 0.5% | 0.5% |
| Squalane | | 5.0% |
| Stearyl alcohol | | 1.5% |
| Tocopherol acetate | 0.5% | |
| Xanthan gum | 0.1% | 0.2% |
| Water | qs 100 | qs 100 |

The purpose of the above description is to illustrate some embodiments of the present invention without implying a limitation. It will apparent to those skilled in the art that various modifications and variations may be made in the apparatus or procedure of the invention without departing from the scope or spirit of the invention.

What is claimed is:

1. A composition for activating at least one of AQP-3, filaggrin or transglutaminase in the skin of an animal, comprising at least one compound from Centella Asiatica selected from the group consisting of madecassoside, terminoloside, asiaticoside, madecassic acid, asiatic acid and mixtures thereof.

2. The composition of claim 1, wherein said composition comprises a composition for enhancing the epidermal functionality.

3. The composition of claim 2, wherein said composition enhances the epidermal functionality by improving the differentiation of keratinocytes.

4. The composition of claim 1 wherein said composition comprises a composition for treatment or prevention of abnormalities of epidermal maturation or keratinisation.

5. The composition of claim 1, wherein said composition comprises a composition for increasing the cohesion of stratum corneum, the epidermal barrier function or skin impermeability.

6. The composition of claim 1, wherein said composition comprises a composition for preventing of epidermal dehydration.

7. The composition of claim 1, wherein said composition comprises a composition for improving epidermal elasticity, flexibility or resistance.

8. The composition of claim 1, wherein said composition comprises a composition for increasing the transfer of water, water-soluble compounds or glycerol through the skin.

9. The composition of claims 1, wherein said composition comprises a composition for activation or regeneration of osmolyte homeostasis, enhancement of keratinocyte drainage or improvement of keratinocyte communication.

10. The composition of claim 1, wherein said composition comprises a composition for the treatments of a skin disease or disorder.

11. The composition of claim 1, wherein said compound comprises a mixture of madecassoside and terminoloside.

12. The composition of claim 11, wherein the weight ratio of said madecassoside to said terminoloside is from about 30:70 to about 70:30.

13. The composition of claim 1, wherein said compound comprises an extract of Centella Asiatica.

14. The composition of claim 1, wherein said extract comprises at least 75% by weight of a mixture of madecassoside and terminoloside.

15. The composition of claim 13, wherein the solvent used to create said extract comprises a mixture of water and ethanol.
